(19) United States
(12) Patent Application Publication
(10) Pub. No.: US 2023/0010826 A1
(43) Pub. Date: Jan. 12, 2023

(54) CONTROL OF A THERAPEUTIC DELIVERY SYSTEM

(71) Applicant: THE BRIGHAM AND WOMEN'S HOSPITAL, INC., Boston, MA (US)

(72) Inventors: Vesela P. Kovacheva, Boston, MA (US); Raphael Cohen, Waltham, MA (US)

(21) Appl. No.: 17/784,631

(22) PCT Filed: Dec. 14, 2020
(86) PCT No.: PCT/US2020/064869

§ 371 Date: Jun. 11, 2022

Related U.S. Application Data
(60) Provisional application No. 62/947,787, filed on Dec. 13, 2019.
(60) Provisional application No. 63/017,705, filed on Apr. 30, 2020.

(51) Int. Cl. A61M 5/142

(52) U.S. Cl. CPC A61M 5/142; A61M 2005/14208; A61M 2205/502; A61M 5/16804

(57) ABSTRACT

System and methods are provided for control of a therapeutic delivery system. A monitoring device includes a sensor for measuring a biometric parameter of a patient. A feature extractor generates a set of features for the patient, each of the set of features being associated with one of the patient and the therapeutic delivery system. A predictive model predicts a future value for the biometric parameter at a given time according to the set of features. A therapeutic delivery system controller determines a desired dosage for the therapeutic according to the predicted future value for the patient parameter.

### RELATED APPLICATIONS

This application claims priority from each of U.S. Provisional Application No. 62/947,787, entitled “PERSONALIZED DRUG INFUSIONS UTILIZING ARTIFICIAL INTELLIGENCE METHODS” and filed 13 Dec. 2019, and U.S. Provisional Application No. 63/017,705, entitled “CONTROL OF A THERAPEUTIC DELIVERY SYSTEM” and filed 30 Apr. 2020. The subject matter of each of these applications is hereby incorporated by reference in its entirety.

### TECHNICAL FIELD

This invention relates to medical systems, and more specifically, to control of a therapeutic delivery system.

### BACKGROUND OF THE INVENTION

Administration and titration of medications to a monitored parameter is one of the major sources for medication errors and risk for patient safety. This process is of particular importance in the operating room (OR) and the intensive care unit (ICU), in which rapid dose adjustments during the continuous administration of medications are often necessary. The process of monitoring key parameters and adjusting the medication accordingly is also one of the most attention-demanding, tedious and time-consuming tasks for an anesthesiologist. For example, in maternal blood pressure (BP) control during cesarean delivery, the current standard of care is that the anesthesiologist monitors maternal BP every minute and administers variable amounts of phenylephrine based on the most recent BP reading, the trend in BP changes and the time elapsed from the spinal injection with the goal of maintaining optimal, target range BP. These decisions are individualized for every patient, since every person reacts to the vasopressor and the local anesthetic differently. This period of hemodynamic instability lasts over twenty minutes. and typically by that time, the baby is delivered.

### SUMMARY OF THE INVENTION

In accordance with one aspect of the present invention, a system is provided for control of a therapeutic delivery system. A monitoring device includes a sensor for measuring a biometric parameter of a patient. A feature extractor generates a set of features for the patient, each of the set of features being associated with one of the patient and the therapeutic delivery system. A predictive model predicts a future value for the biometric parameter at a given time according to the set of features. A therapeutic delivery system controller determines a desired dosage for the therapeutic according to the predicted future value for the patient parameter.

In accordance with another aspect of the invention, a method is provided for controlling a therapeutic delivery system according to a monitored patient parameter. A patient parameter is monitored. A set of features is extracted for a patient including at least the monitored patient parameter. A future value for the monitored patient parameter is predicted at a predictive model according to the extracted set of features. A dosage is selected for the therapeutic delivery system according to the predicted future value of the monitored patient parameter.

In accordance with still another aspect of the present invention, a system is provided for control of a therapeutic delivery system. A monitoring device includes a plurality of sensors for measuring a plurality of patient parameters of a patient. The plurality of patient parameters includes a target patient parameter. A feature extractor generates a set of features for the patient, each of the set of features being associated with one of the patient and the therapeutic delivery system. The set of features includes a feature representing one of a current and past dosage of the therapeutic being provided by the therapeutic delivery system and at least two features derived from the plurality of patient parameters. A predictive model predicts a future value for the target patient parameter at a given time according to the set of features. A therapeutic delivery system controller determines a desired dosage for the therapeutic according to the predicted future value for the target patient parameter.

### BRIEF DESCRIPTION OF THE DRAWINGS

FIG. 1 illustrates a system for determining a dosage for administration of medication via a therapeutic delivery system in accordance with an aspect of the present invention;

FIG. 2 illustrates an example implementation of a system for controlling a therapeutic delivery system via a prediction of a future value of a patient parameter;

FIG. 3 illustrates a method for controlling a therapeutic delivery system according to a monitored patient parameter;

FIG. 4 illustrates another method for controlling a therapeutic delivery system according to a monitored patient parameter; and

FIG. 5 is a schematic block diagram illustrating an exemplary system of hardware components.

### DETAILED DESCRIPTION

As used in this application, a “patient parameter” refers to medically relevant data associated with the patient. This can include, but is not limited to, patient characteristics, such as age, sex, height, and weight; laboratory values such as blood glucose level, blood chemistry panels, complete blood counts, blood gas levels, urinalysis, culture results; and clinical measurements such as blood pressure, heart rate, oxygen saturation, exhaled gases concentration, and ventilation parameters, and medical history, including past values for clinical measurements and patient characteristics, diagnoses of medical conditions, allergies, and current and past therapeutic interventions performed on the patient.

A “therapeutic,” is a medication or another therapeutic substance, such as blood, intravenous fluid, or inhaled gas.

A “therapeutic delivery system”, as used herein, is a device, controllable via an electrical signal, that provides a therapeutic to a patient.

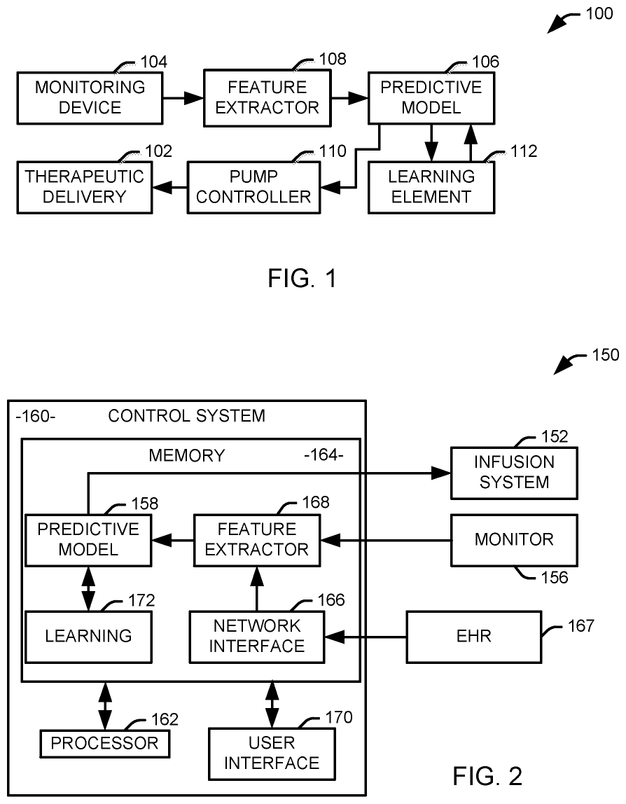

FIG. 1 illustrates a system 100 for determining a dosage for administration of a therapeutic via a therapeutic delivery system 102 in accordance with an aspect of the present invention. It will be appreciated that the therapeutic delivery system 102 can be controlled directly to provide the determined dosage to a patient or an appropriate dosage can be provided to a human operator for verification before inputting the dosage into the therapeutic delivery system 102. The system 100 includes a monitoring device 104 that include a sensor for measuring a biometric parameter of a patient. The specific implementation of the monitoring device 104 will vary with the monitored patient parameter and the environment in which the therapeutic delivery system 102 is employed.

For example, in an operating room environment, in which the therapeutic delivery system 102 is used to deliver a therapeutic, the monitoring device 104 can include, for example, one or more sensors for measuring the volume and content of any of exhaled air, pulse oximeters, heart rate monitors, sphygmomanometers, and other sensors for monitoring the physiological function of the patient in the form of patient parameters. For example, the therapeutic delivery system 102 could include an infusion pump to deliver a fluid or medication to the patient or an anesthesia machine for providing inhalational anesthetic to the patient, with the therapeutic delivery system receiving input from the blood pressure in the first case and the bispectral index or the continuous measurement of the end-tidal inhalational anesthetic concentration, in the second case. In another example, the monitoring device 104 can be a non-invasive blood pressure monitor carried or worn by a patient to obtain regular measurements of a blood pressure of the patient. In this instance, the therapeutic delivery system 102 can be used to deliver a medication to control the blood pressure of the patient. In yet another example, the monitoring device 104 can be a carried, worn, or implantable device for monitoring a blood glucose of a patient, and the therapeutic delivery system 102 can be used to deliver insulin to the patient.

It will be appreciated that administered therapeutics do not generally act instantaneously, and that both the magnitude and timing of effects from therapeutics can vary with patients. In addition, making the action of the therapeutic delivery system 102 directly dependent on the data received from the monitoring device 104 may not provide optimal results for all or even most patients. The system predicts a future value for a patient parameter that can be used to derive an appropriate dosage for the therapeutic at a predictive model 106. In practice, the predictive model 106 can be provided with one or more features derived from the monitored patient parameters and output a result representing a value of one of the monitored patient parameters after a predetermined amount of time. In addition to the monitored patient parameters, clinical data can also be used for generating features and predicting this value. Such clinical data can include, for example, the patient's height, weight, and medical history (e.g., existing conditions and other medications). The predictive model 106 can be implemented, for example, as machine-readable instructions stored in a tangible memory and executed by an associated processor, either on a device local to the therapeutic delivery system 102, networked or a remote system connected via an Internet connection.

A feature extractor 108 can be provided with the data from the monitoring system 104 and any relevant clinical data. The feature extractor 108 extracts a plurality of features, which can be categorical, discrete, and continuous parameters representing the monitored patient parameters and the clinical data. In one example, the parameters can include descriptive statistics, such as measures of central tendency (e.g., median, mode, arithmetic mean, or geometric mean) and measures of deviation (e.g., range, interquartile range, variance, standard deviation, etc.) of time series of the monitored patient parameters. Additionally or alternatively, the patient parameters can be used to assign a plurality of categorical parameters to the user according to thresholds for monitored patient parameters or rule sets that act upon time series of values for the monitored patient parameters, for example, representing the presence or absence of a given condition or behavior. In one example, any of a current, past, or predicted BP can also be utilized as a feature at the predictive model.

The predictive model 106 can utilize one or more pattern recognition algorithms, each of which analyze the extracted features or a subset of the extracted features to assign a continuous or categorical parameter to the user. In one example, the assigned parameter can represent a continuous value representing one of the monitored patient parameters. It will be appreciated, however, that additional or alternative features can be used in the analysis and that the index can be replaced with a categorical classification (e.g., “normal”, “low”, “high”) in some implementations.

Where multiple classification or regression models are used, an arbitration element can be utilized to provide a coherent result from the plurality of models. The training process of a given classifier will vary with its implementation, but training generally involves a statistical aggregation of training data into one or more parameters associated with the output class. For rule-based models, such as decision trees, domain knowledge, for example, as provided by one or more human experts, can be used in place of or to supplement training data in selecting rules for classifying a user using the extracted features. Any or a combination of a variety of techniques can be utilized for the classification algorithm, including support vector machines (SVMs), regression models, self-organized maps, fuzzy logic systems, data fusion processes, boosting and bagging methods, rule-based systems, or artificial neural networks (ANNs). In addition, these techniques can be used in combination with classical statistical techniques and rule-based techniques.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. In one implementation, the SVM can be implemented via a kernel method using a linear or non-linear kernel.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a rectified linear unit. A final layer of nodes provides the confidence values for the output classes of the ANN, with each node having an associated value representing a confidence for one of the associated output classes of the classifier.

Many ANN classifiers are fully-connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a type of recurrent neural networks, which makes it easier to remember past data in memory.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used.

In the illustrated example, the output of the predictive model 106 is provided to a therapeutic delivery system controller 110 that uses the output of the predictive model 106 to determine an optimal dosage to be provided by the therapeutic delivery system 102. It will be appreciated that this can be done as a feedback mechanism, where a dosage is adjusted in an appropriate direction when a predicted value for the monitored patient parameter departs a desired range or when a categorical output from the predictive model 106 indicates that a change is necessary. Alternatively, the predictive model 106 can be employed to evaluate a plurality of possible dosages, with a dosage selected that provides a predicted value for the monitored patient parameter that is most similar to an optimal value. The therapeutic delivery system controller 110 can then be provided with the selected dosage for the patient.

It will be appreciated that the monitored patient parameter can, and generally will, be monitored after the prediction at the predictive model 106. Accordingly, a learning element 112 can store the features used for the prediction, the output of the predictive model 106, and the actual value for the monitored patient parameter. The learning element 112 can compare the predicted value generated at the predictive model 106 to generate an error for each output of the predicted model. This error can be tracked, and stored data at the learning element 112 can be used to retrain or otherwise refine the predictive model 106 whenever a threshold error is recorded. The threshold error can represent, for example, a single error value or a windowed sum of the squared error across a series of measurements.

FIG. 2 illustrates an example implementation of a system 150 for controlling a therapeutic delivery system via a prediction of a future value of a patient parameter. Examples of therapeutics that could be provided via an infusion system include medications used for induction of general anesthesia, such as propofol, or vasopressors such as phenylephrine. In the illustrated example, an infusion system 152 is used to provide phenylephrine to the patient to control blood pressure (BP) of the patient during cesarean delivery under spinal anesthesia. It will be appreciated, however, that the system 150 could be used in any surgery where tight control of blood pressure or depth of anesthesia (e.g., as measured by a bispectral index) is needed, especially obstetric, cardiac, ENT and neurosurgery, and the therapeutic delivery system could be, for example, the illustrated infusion system 152 and/or an anesthesia system delivering anesthetic gas. While the system 150 would not be expected to replace the judgement of a trained anesthesiologist, it could allow for automation in a tedious task, help avoid drug administration errors, and allow the anesthesiologist to direct attention to more important tasks.

Maternal BP control during delivery is one of the most important factors for the well-being of mother and baby. Low maternal BP has been associated with nausea, vomiting, dizziness and, in rare cases, with loss of consciousness and stroke. In the neonate, low maternal BP can lead to acidosis and hypoxia, which can correlate with poor outcome. Spinal anesthesia results in a profound drop in maternal BP due to sympathetic blockade. In order to prevent that, anesthesiologists administer vasopressors preemptively. The exact type, amount, and timing of vasopressor administration have been extensively studied, however, there is no clear predictive model. Surprisingly, the degree of hypotension does not appear to depend on maternal BMI, age, gestational age and number and weight of the fetuses.

The current standard of care is manual calculation by the anesthesiologist of the phenylephrine doses every minute, which is prone to error and results in high rate of nausea, lightheadedness, and suboptimal blood pressure control. The system 150 uses input parameters from anesthesia monitors 156, processes the information in real time via a machine learning model 158, and directs the infusion system 152 to administer medication to the patient. The system 150 receives periodic real-time maternal systolic blood pressure (SBP) measurements obtained from the anesthesia monitors 156, utilizes the machine learning model 158 to generate an amount of phenylephrine, and direct the infusion system 152 to administer it to the patient. The predictive model 158 calculates the amount of phenylephrine needed based not only on the current value of the SBP, but also on the SBP trend for that particular patient, the time elapsed from the administration of the spinal, the pharmacokinetics of the drug, and the predicted future values of one or more of these parameters. While autonomous, the system 150 is intended to operate with an anesthesiologist present at all times and ultimately responsible for the patient care.

In the example of FIG. 2, the system 150 includes a control system 160 for the infusion system 152. The control system 160 can include one or more processors 162 and a memory 164. It will be appreciated that the memory 164 can comprise one or more discrete units of physical memory operatively connected to process to store data and machine-readable instructions that can be executed by the processor 162. For example, the memory 164 can comprise physical memory, which can reside on the processor 162 (e.g., processor memory), random access memory or other physical storage media (e.g., CD-ROM, DVD, flash drive, hard disc drive, etc.) or a combination of different memory devices that can store the executable instructions. The data utilized for implementing the systems and methods described herein can also be stored in the memory 164 or in some other arrangement of one or more memory structures that are accessible for use by the control system 160.

The control system 160 can be connected to a network via a network interface 166 to provide for communication between the control system 160 and various services, devices, and data stores that contain patient data. The network can be a local area network, a wide area network, or a combination of different various network topologies, which may include physical transmission media (e.g., electrically conductive, optical fiber media or the like) and/or wireless communications media, that can be utilized for communicating information. The network, or at least a portion of the methods and functions implemented thereby, can operate in a secure manner (e.g., behind a firewall separated from public networks) and/or utilize encryption for data communications. In one example, the control system 160 retrieves patient data from an electronic health records (EHR) database 167 via the network interface 166. In general, the patient data collected from the electronic health records database includes relatively static characteristics of the patient, such as demographic information, height, weight, baseline renal function, a baseline blood pressure, chronic conditions, current medications, allergies, and other medical history. If an EHR is not available, these parameters may be entered manually. Accordingly, these parameters can be retrieved once at the start of a procedure.

The control system 160 receives patient parameters from the anesthesia monitor 156, which can include, for example, systolic blood pressure, mean arterial blood pressure, heart rate, temperature, values extracted from an electrocardiogram, respiratory rate, end tidal inhalational anesthetic concentration, a fraction of inspired oxygen, tidal volume, airway pressure, oxygen saturation and other respiratory parameters. The infusion system 152 can also communicate the current dosages of therapeutics being provided to the patient. In the illustrated system, these values are received from the anesthesia monitor 156 each minute.

The patient parameters are then provided to a feature extractor 168 that extracts a set of features for predicting a next measurement of the systolic blood pressure (SBP) at the predictive model 158. In the illustrated example, the extracted features can include a baseline blood pressure for the patient, a baseline heart rate, a change in the systolic blood pressure from the baseline value, the dosage of a spinal anesthetic (e.g., bupivacaine), and the dosage of the vasopressor provided via the infusion system 152 (e.g., phenylephrine). These features are then provided to the predictive model 158. It will be appreciated that these features can be generated as a time series, with both past and current values provided to the predictive model. In one example, the predictive model 158, in this example, generated as a linear mixed-effects model with a Gaussian random intercept to prognosticate the next SBP during administration of spinal anesthesia. The model fit was assessed by calculating the marginal pseudo-R² (the variance explained by fixed effects) and conditional pseudo-R² (the variance explained by entire model) for generalized mixed-effect models, and it was determined that a change in SBP from baseline at the current time point explains 68.8% of the variability in change in SBP from baseline at the next time point.

In another example. the model is implemented as a multi-parameter partial differential equation to predict a patient's systolic blood pressure at the next time step given temporally-spaced vital signs readings. The model encodes expected physiological patient reactions into its terms for a general differential equation solution and live-fitted to the individual patient during use. Hyperparameter values, representing expected physiological patient reactions, are initialized to produce a behavior that is generally amenable with the anesthesiologists' expectations. These values can be adjusted by the anesthesiologist at a user interface 170 either before administration of the therapeutic begins, to account for specific characteristics of the patient, or during administration of the therapeutic, to adjust for unexpected reactions of the patient to the therapeutic or other circumstances. In addition, the model can detect deviations from the expected pattern and also self-adjust or adjust in addition to the physician input. The mean-squared error is assigned as the cost function between the model's prediction and the truth values at each predicted point. The gradient, derived from predefined physiological behaviors, and hyperparameter updates, limited by predefined physiological constraints, can be used to update each of the parameters in the model through a non-convex optimization task. This provides a differential equation solution that is amenable to high-quality predictions of blood pressure within patients following normal behavior.

With the forecast of the future patient blood pressure, the model itself can then be used to predict an amount of phenylephrine to dose to the patient to keep their blood pressure stable within a target blood pressure range. The target blood pressure can be selected automatically from known characteristics of the patient or entered by a physician at the user interface 170. The stable point can be temporally adjusted based on a user-defined threshold which, given the recorded history of the patient, causes the model to make predictions that will keep the patient at the target blood pressure not just at the next time step, but multiple steps in the future, taking into account the amount of phenylephrine in the system at each time point.

The model can also include a learning component 172 that refines the hyperparameters of the model on a patient-by-patient basis using a learning function that accepts both the model's parameters, predictions, and the patient information. In particular, the learning component 172 can store each of the features used for the prediction, the predicted future value, and an actual value for the monitored patient parameter at the given time and compare the predicted future value to the actual value to generate an error value for the predicted future value. This may improve the model's descriptive abilities for each patient, producing a better blood pressure prediction, and with it an improved prediction of phenylephrine.

To further account for variance among patients, the model can alert the user if the patient behavior is unusual, or if the model is making unexpected predictions. In one example, the learning element stores a series of error values associated with a corresponding series of predicted future values and provides an alert if the error values exceed a threshold value, either at any point or for a predetermined period of time. Similarly, the learning element can provide an alert if a monitored patient parameter deviates from a desired range for a predetermined period of time. For example, the model can provide alerts when the patient's blood pressure falls outside ninety-five percent of all patients within the training set, or if it rises above or below thresholds at the attending anesthesiologists' discretion. The model will also provide alerts if the predicted phenylephrine dosage rises above a threshold set by the anesthesiologist. The model may have an upper bound on the amount of phenylephrine it is able to administer at the infusion system 152.

The user interface 170 can include a display to display the dosage of the therapeutic, the monitored patient parameter, and the predicted value for the monitored patient parameter to a user as well as an input device, such as a keyboard, touchscreen, or mouse. The user interface 170 can be employed by the user to adjust the hyperparameters of the model as to tune the model to the physiological reactions of a given patient to the therapeutic. For example, patients with hypertensive disorders of pregnancy are very sensitive to vasopressors and need lower doses of phenylephrine, compared to healthy patients, to maintain the same blood pressure. As this diagnosis is typically known in advance, the physician can adjust the hyperparameter that represents the patient's sensitivity to phenylephrine so that the system does not administer a dose that would have been appropriate for a healthy patient, but too high for hypertensive patient. In one example, each hyperparameter of the predictive model 158 is represented by a slider bar on the display that can be moved by an anesthesiologist to dynamically adjust the hyperparameters.

The learning model 172 can also include a pattern recognition component that identifies patterns of errors in the model associated with certain disorders. In the instant example, various patterns of errors can indicate that the patient may be at risk for developing preeclampsia after the procedure for which the therapeutic is being administered. Along with alerting a physician, the learning model 172 can be configured to automatically adjust one or more hyperparameters of the model to account for the identified condition or to switch the predictive model 158 entirely to a different model trained on individuals with the identified condition.

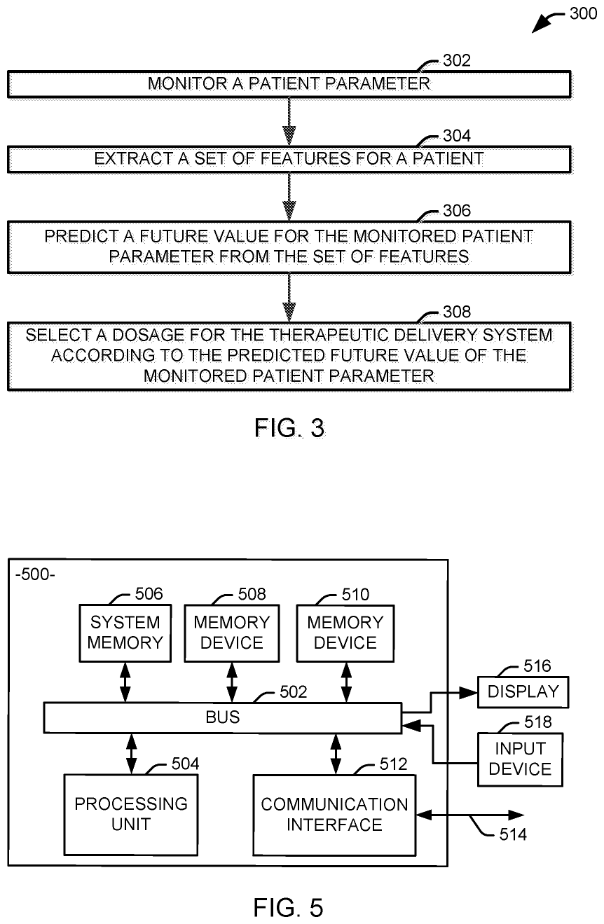

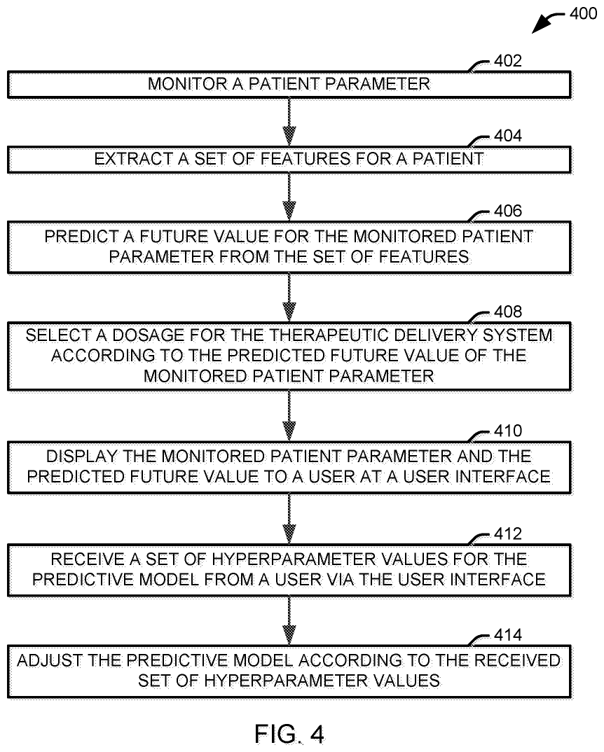

In view of the foregoing structural and functional features described above, methods in accordance with various aspects of the present invention will be better appreciated with reference to FIGS. 3 and 4. While, for purposes of simplicity of explanation, the methods of FIGS. 3 and 4 are shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a method in accordance with an aspect the present invention.

FIG. 3 illustrates one method 300 for controlling a therapeutic delivery system according to a monitored patient parameter. The method 300 utilizes a predictive model to predict a future value for the monitored patient parameter and determines a dosage for therapeutic delivery from that parameter. It will be appreciated that , in practice, multiple patient parameters can be monitored for use in providing information to the predictive model and controlling the therapeutic delivery system, but in the illustrated method, the parameter predicted by the model is referred to as the “monitored patient parameter.”

At 302, the monitored patient parameter is monitored. At 304, a set of features is extracted for a patient. The set of features can include past and current values for the monitored patient parameter, past and current values for other monitored patient parameters, information extracted from a patient record in an electronic health record database, current, past, or predicted future dosages for the therapeutic being delivered by the system, and current, past, and predicted dosages for other therapeutics administered to the patient. In one example, the set of features includes a feature representing a current, past, or predicted future dosage of the therapeutic being provided by the therapeutic delivery system and at least two features derived from the plurality of patient parameters.

At 306, a future value for the monitored patient parameter is predicted at a predictive model according to the extracted set of features. In one example, the predictive model is implemented as a multi-parameter partial differential equation. In one implementation, the predictive model includes a learning element that stores the set of features used for the prediction, the predicted future value, and an actual value for the monitored patient parameter at the given time and compares the predicted future value to the actual value to generate an error value for the predicted future value. The stored data can be used in adjusting a set of hyperparameters associated with the model over time or as additional training samples for refining the model.

At 308, a dosage for the therapeutic delivery system is selected according to the predicted future value of the monitored patient parameter. It will be appreciated that this can be done as a feedback mechanism, where a dosage is adjusted in an appropriate direction when a predicted value for the monitored patient parameter departs a desired range or when a categorical output from the predictive model indicates that a change is necessary. Alternatively, the predictive model can be employed to evaluate a plurality of possible dosages, with a dosage selected that provides a predicted value for the monitored patient parameter that is most similar to an optimal value.

FIG. 4 illustrates another method 400 for controlling a therapeutic delivery system according to a monitored patient parameter. As in FIG. 3, it will be appreciated that multiple patient parameters can be monitored for use in providing information to a predictive model to control the therapeutic delivery system, but the parameter predicted by the model is referred to here as the “monitored patient parameter.” At 402, the monitored patient parameter is monitored. At 404, a set of features is extracted for a patient. The set of features can include past, current, or predicted future values for any patient parameters monitored during therapeutic delivery or dosages of provided therapeutics as well as information extracted from a patient record in an electronic health record database.

At 406, a future value for the monitored patient parameter is predicted at a predictive model according to the extracted set of features. At 408, a dosage for the therapeutic delivery system is selected according to the predicted future value of the monitored patient parameter. It will be appreciated that this can be done as a feedback mechanism, where a dosage is adjusted in an appropriate direction when a predicted value for the monitored patient parameter departs a desired range or when a categorical output from the predictive model indicates that a change is necessary. Alternatively, the therapeutic delivery system controller can employ a model or function that accepts one or more of the extracted features and predicts a therapeutic dosage. In one example, that function employs the predictive model to evaluate a plurality of possible dosages, with a dosage selected that provides a predicted value for the monitored patient parameter that is most similar to an optimal value.

At 410, each of the monitored patient parameter and the predicted future value are displayed to a user at a user interface. At 412, a set of hyperparameter values are received for the predictive model from a user via the user interface. In one example, the user can use an input device to change the positions of various sliders or scroll bars to select values for the hyperparameters. The hyperparameters can represent, for example, the patient's expected sensitivity to the therapeutic or other therapeutics provided to the patient. The user can also select target values or ranges for the monitored parameter using this interface. In general, the user may opt to adjust the hyperparameter values during a procedure when the model is performing sub-optimally for a given patient. At 414, the predictive model is adjusted according to the received set of hyperparameter values.

It will be appreciated that the method of FIG. 4 is of particular value when using a model that is transparent to the user, in some instances such that the values for the hyperparameters represent known physiological parameters. This allows a clinician to adjust the model to match a physiological or clinical property of a patient, such as a sensitivity to a particular therapeutic, in accordance with clinical judgment. Accordingly, the method 400 can utilize an explainable artificial intelligence (xAl) approach to implement the predictive model. For example, the current partial differential equation model yields an inherently explainable framework to interpret model decision making, which can be displayed to a physician. Other examples of xAl approaches include deep Taylor Decomposition, Local Interpretable Model-Agnostic Explanations (LIME), and Shapely Additive Explanations (SNAP). In such an approach, the initial values for the hyperparameters can be determined for a given patient from the dataset used for training the predictive model, with the xAl component providing an explanation of the reasoning used by the model to generate the hyperparameters for review by the clinician before adjusting the parameters. Accordingly, the performance of the model can be improved over approaches where the hyperparameters are modified by the user or the hyperparameter optimization is purely automated.

FIG. 5 is a schematic block diagram illustrating an exemplary system 500 of hardware components capable of implementing examples of the systems and methods disclosed herein. The system 500 can include various systems and subsystems. The system 500 can be a personal computer, a laptop computer, a workstation, a computer system, an appliance, an application-specific integrated circuit (ASIC), a server, a server BladeCenter, a server farm, etc.

The system 500 can include a system bus 502, a processing unit 504, a system memory 506, memory devices 508 and 510, a communication interface 512 (e.g., a network interface), a communication link 514, a display 516 (e.g., a video screen), and an input device 518 (e.g., a keyboard, touch screen, and/or a mouse). The system bus 502 can be in communication with the processing unit 504 and the system memory 506. The additional memory devices 508 and 510, such as a hard disk drive, server, standalone database, or other non-volatile memory, can also be in communication with the system bus 502. The system bus 502 interconnects the processing unit 504, the memory devices 506, 508, and 510, the communication interface 512, the display 516, and the input device 518. In some examples, the system bus 502 also interconnects an additional port (not shown), such as a universal serial bus (USB) port.

The processing unit 504 can be a computing device and can include an application-specific integrated circuit (ASIC). The processing unit 504 executes a set of instructions to implement the operations of examples disclosed herein. The processing unit can include a processing core.

The additional memory devices 506, 508, and 510 can store data, programs, instructions, database queries in text or compiled form, and any other information that may be needed to operate a computer. The memories 506, 508 and 510 can be implemented as computer-readable media (integrated or removable), such as a memory card, disk drive, compact disk (CD), or server accessible over a network. In certain examples, the memories 506, 508 and 510 can comprise text, images, video, and/or audio, portions of which can be available in formats comprehensible to human beings.

Additionally or alternatively, the system 500 can access an external data source or query source through the communication interface 512, which can communicate with the system bus 502 and the communication link 514.

In operation, the system 500 can be used to implement one or more parts of a system for controlling the operation of a therapeutic delivery system in accordance with the present invention. Computer executable logic for implementing the system resides on one or more of the system memory 506, and the memory devices 508 and 510 in accordance with certain examples. The processing unit 504 executes one or more computer executable instructions originating from the system memory 506 and the memory devices 508 and 510. The term “computer readable medium” as used herein refers to a medium that participates in providing instructions to the processing unit 504 for execution. This medium may be distributed across multiple discrete assemblies all operatively connected to a common processor or set of related processors. Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. A system comprising:
a therapeutic delivery system that delivers a therapeutic to a patient;
a monitoring device that includes a sensor for measuring a biometric parameter of a patient;
a feature extractor that generates a set of features for the patient, each of the set of features being associated with one of the patient and the therapeutic delivery system;
a predictive model that predicts a future value for the biometric parameter at a given time according to the set of features; and
a therapeutic delivery system controller that determines a desired dosage for the therapeutic according to the predicted future value for the patient parameter.

2. The system of claim 1, wherein the therapeutic delivery system is an infusion system, and the patient parameter is a blood pressure of the patient.

3. The system of claim 1, wherein the therapeutic delivery system is an insulin pump, and the patient parameter is a blood glucose of the patient.

4. The system of claim 1, wherein the sensor is a first sensor of a plurality of sensors and the patient parameter is a first patient parameter of a plurality of patient parameters, the feature extractor generating at least two of the set of features from the plurality of patient parameters.

5. The system of claim 1, wherein the set of features includes one of a current and a past dosage associated with the therapeutic delivery system.

6. The system of claim 1, further comprising a learning element that stores the set of features used for the prediction, the predicted future value, and an actual value for the monitored patient parameter at the given time and compares the predicted future value to the actual value to generate an error value for the predicted future value.

7. The system of claim 6, wherein the learning element stores a series of error values associated with a corresponding series of predicted future values and provides an alert if the error values exceed a threshold value for a predetermined period of time.

8. The system of claim 6, wherein the learning element provides an alert if the patient parameter deviates from a desired range for a predetermined period of time.

9. The system of claim 1, further comprising a user interface that displays the patient parameter and the desired dosage to a user and allows a user to change at least one parameter associated with the predictive model.

10. The system of claim 1, wherein the set of features comprises at least one feature represented as a time series, and the predictive model is implemented as a multi-parameter partial differential equation.

11. The system of claim 10, wherein the multi-parameter partial differential equation includes a set of hyperparameters representing physiological patient reactions, the hyperparameters being adjustable by a user via a user interface.

12. The system of claim 1, further comprising a network interface that provides patient data from an electronic health records (EHR) database to the feature extractor, at least one features of the set of features being determined from the patient data.

13. A method for controlling a therapeutic delivery system according to a monitored patient parameter, the method comprising:
monitoring a patient parameter;
extracting a set of features for a patient including at least the monitored patient parameter;
predicting a future value for the monitored patient parameter at a predictive model according to the extracted set of features; and
selecting a dosage for the therapeutic delivery system according to the predicted future value of the monitored patient parameter.

14. The method of claim 13, further comprising:
displaying each of the monitored patient parameter and the predicted future value to a user at a user interface;
receiving a set of hyperparameter values for the predictive model from a user via the user interface; and
adjusting the predictive model according to the received set of hyperparameter values.

15. The method of claim 13, further comprising:
storing the set of features used for the prediction, the predicted future value, and an actual value for the monitored patient parameter at the given time; and
comparing the predicted future value to the actual value to generate an error value for the predicted future value.

16. The method of claim 13, wherein the monitoring a patient parameter comprises monitoring a plurality of patient parameters and the set of features includes a feature representing a current dosage and a past dosage of the therapeutic being provided by the therapeutic delivery system and at least two features derived from the plurality of patient parameters.

17. The method of claim 13, wherein the predictive model is implemented as a multi-parameter partial differential equation.

18. A system comprising:
a therapeutic delivery system that delivers a therapeutic to a patient;
a monitoring device that includes a plurality of sensors for measuring a plurality of patient parameters of a patient, the plurality of patient parameters comprising a target patient parameter;
a feature extractor that generates a set of features for the patient, each of the set of features being associated with one of the patient and the therapeutic delivery system and the set of features including a feature representing one of a current dosage and a past dosage of the therapeutic being provided by the therapeutic delivery system and at least two features derived from the plurality of patient parameters;
a predictive model that predicts a future value for the target patient parameter at a given time according to the set of features; and
a therapeutic delivery system controller that determines a desired dosage for the therapeutic according to the predicted future value for the target patient parameter.

19. The system of claim 18, wherein the set of features comprises at least one feature represented as a time series, and the predictive model is expressed as a multi-parameter partial differential equation including a set of hyperparameters representing physiological patient reactions, the hyperparameters being adjustable by a user via a user interface.

20. The system of claim 18, further comprising a learning element that stores the features used for the prediction, the predicted future value, and an actual value for the target patient parameter at the given time, compares the predicted future value to the actual value to generate an error value for the predicted future value, stores a series of error values associated with a corresponding series of predicted future values, and provides an alert if the error values exceed a threshold value for a predetermined period of time.
